(19) United States
(12) Patent Application Publication
(10) Pub. No.: US 2025/0152645 A1
(43) Pub. Date: May 15, 2025

(54) SINGLE CELL PROTEIN PRODUCTS

(71) Applicant: DSM IP ASSETS B.V., Heerlen (NL)

(72) Inventors: Carol ROA-ENGEL, Kaiseraugst (CH); Mickel Leonardus August JANSEN, Kaiseraugst (CH); Evert Tjeerd VAN RIJ, Kaiseraugst (CH); Hendrik Jan NOORMAN, Kaiseraugst (CH); Ester SANTIGOSA CULI, Kaiseraugst (CH)

(21) Appl. No.: 18/838,938

(22) PCT Filed: Feb. 17, 2023
(86) PCT No.: PCT/EP2023/054011

§ 371 Date: Aug. 15, 2024

Related U.S. Application Data
(60) Provisional application No. 63/311,208, filed on Feb. 17, 2022.

(51) Int. Cl. A61K 36/064; A23K 10/16; A23K 20/147; A23K 50/80; C12N 1/16; C12R 1/645

(52) U.S. Cl. CPC A61K 36/064; A23K 10/16; A23K 20/147; A23K 50/80; C12N 1/165; C12R 2001/645

(57) ABSTRACT

The present invention relates to an animal feed comprising up to 20% (w/w) yeast single cell protein product, wherein the yeast single cell protein product comprises Saccharomycesales yeast cells, as well as to the use of said animal feed for feeding an animal and/or for increasing body weight of an animal.

### I. FIELD OF THE INVENTION

The present invention relates to an animal feed comprising up to 20% (w/w) yeast single cell protein product, wherein the yeast single cell protein product comprises Saccharomycesales yeast cells, as well as to the use of said animal feed for feeding an animal and/or for increasing body weight of an animal.

### II. BACKGROUND

The world's population is growing and so is the demand for food, including the demand for meat, dairy products and seafood. Animal feeding requires considerable amounts of protein for ensuring optimal growth and fattening of animals with a major source of protein being currently constituted by plants in traditional breeding. However, plant-derived proteins tend to be poorly converted into animal-derived protein. Furthermore, plant-derived protein production is associated with potential ethical conflicts between food and feed production. Also seafood has been experiencing an increasing demand as it is considered to confer health benefits when included in the regular diet. To meet this demand, aquaculture provides currently almost half of the world's fish for human consumption. Feeding in aquaculture is increasingly based on a combination of plant-derived protein sources and alternatives like fish meal. However, fish meal is often obtained by catching wild fish stocks and hence, may not be considered a sustainable alternative either. Also, the culture of fish and crustaceans for example is becoming increasingly intensive and resource consuming. Thus, breeders and hence, the animal-protein derived protein production industry, including the aquaculture industry, need new protein sources to keep growing at high speed. However, health and welfare of the animals has to be ensured and their growth optimized, whilst moving away from traditional protein sources and especially also wild marine ingredients. Thus, to meet the demand for meat, dairy products and seafood in view of current and expected consumption levels, alternative protein sources in animal feeding are required.

Besides plant- and animal-derived protein, a further source of proteins has been identified, namely microbial-derived proteins. Said single cell proteins (SCPs) can be produced using fungi, algae and/or bacteria that offer the ability of large scale culturing at comparatively low cost. Thus, use of SCP products has been exploited in animals feed, e.g. for poultry, calves, pigs, and fish, but also, e.g., in the leather and paper industry. However, SCP product production still faces several challenges, like availability and sustainability of feedstock, like waste-based feedstock. Thus, also feed millers and farmers still require alternative solutions for protein production to be able to provide low carbon footprint diets while ensuring an environmental-friendly use of the planet's resources.

Hence, there is still a need to have at hand an animal feed that comprises protein from an alternative protein source and thus, protein that is neither animal-nor plant-derived.

### III. SUMMARY

Thus, the present invention aims at providing an animal feed comprising with up to 20% (w/w) single cell protein an alternative protein source that can supplement or even replace currently used animal-and/or plant-derived protein sources in animal feed. Thus, the increasing demand for alternative feed solutions can be met, while ensuring sustainability.

In particular, the present invention relates to an animal feed comprising up to 20% (w/w), preferably up to 10% (w/w), yeast single cell protein (SCP) product, wherein the yeast single cell protein product comprises Saccharomycesles yeast cells.

Preferably, the yeast SCP product comprises Saccharomycesles yeast cells that are not Saccharomyces cerevisiae yeast cells, and/or wherein the yeast SCP product does not comprise Saccharomyces cerevisiae yeast cells.

Preferably, the SCP product comprises ethanol fed Saccharomycesales yeast cells.

Preferably, the Saccharomycesales yeast cells are yeast cells from the genus Cyberlindnera, Kluyveromyces, Wickerhamomyces, Pichia or Yarrowia, preferably from the genus Cyberlindnera or Kluyveromyces or Wickerhamomyces, preferably wherein the Saccharomycesles yeast cells are from Cyberlindnera jadinii, Kluyveromyces lactis, Wickerhamomyces anomalus, Pichia anomala or Yarrowia lipolytica, preferably from Cyberlindnera jadinii, Kluyveromyces lactis, Wickerhamomyces anomalus, preferably wherein the Saccharomycesles yeast cells are or are derived from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950, Kluyveromyces lactis CBS 2896, Wickerhamomyces anomalus CBS 2576 or Yarrowia lipolytica CBS 7504, preferably from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950 or Kluyveromyces lactis CBS 2896.

Preferably, the Saccharomycesales yeast cells are capable of producing with ethanol as carbon source 34% (w/w) or more protein per gram dry weight of said Saccharomycesles yeast cells, preferably 41% (w/w) or more protein per gram dry weight of said Saccharomycesles yeast cells, more preferably 42.5% (w/w) or more protein per gram dry weight of said Saccharomycesles yeast cells.

Preferably, the yeast SCP product comprises all essential amino acids.

Preferably, the Saccharomycesales yeast cells are not genetically engineered.

Preferably, the yeast SCP product comprises 34% (w/w) or more protein per gram dry weight of Saccharomycesles yeast cells, preferably 41% (w/w) or more protein per gram dry weight of Saccharomycesles yeast cells, more preferably 42.5% (w/w) or more protein per gram dry weight of Saccharomycesales yeast cells.

Preferably, the yeast SCP product comprises dried Saccharomycesles yeast cells, preferably wherein said dried Saccharomycesles yeast cells are intact or disrupted or a mixture of intact and disrupted cells.

Preferably, the animal feed further comprises i) further less than 25% (w/w) plant-based protein products, ii) further 5% (w/w) or less fish meal, iii) no fish meal, and/or iv) from 1 to 25% (w/w) oil.

Preferably, the animal feed is a feed for pigs, cows, or sheep.

Preferably, the animal feed is a feed for aquatic species, wherein said aquatic species are preferably selected from crustaceans or fish, preferably wherein said crustaceans are shrimps and/or preferably wherein said fish are warm water fish or cold water fish, preferably wherein said warm water fish are selected from tilapia, seabream, seabass, or carp and/or preferably wherein said cold water fish are selected from salmon or rainbow trout.

The present invention also relates to the use of the animal feed according to the present invention for feeding an animal.

The present invention also relates to the use of the animal feed according to the present invention for increasing body weight of an animal.

Preferably, the animal is a pig, cow, or sheep or said animal are aquatic species, preferably wherein said aquatic species are selected from crustaceans or fish, preferably wherein said crustaceans are shrimps and/or preferably wherein said fish are warm water fish or cold water fish, preferably wherein said warm water fish are selected from tilapia, seabream, seabass, or carp and/or preferably wherein said cold water fish are selected from salmon or rainbow trout.

### IV. DETAILED DESCRIPTION

The technical problem is solved by the subject-matter as defined in the claims, described in the description, exemplified in the Examples and illustrated in the Figures.

In particular, it was surprisingly found that Saccharomycesles yeast cells, when grown with ethanol as carbon source, produce high protein, i.e. 34% (w/w) or more protein per gram dry weight of such Saccharomycesles yeast cells, preferably 41% (w/w) or more protein per gram dry weight of such Saccharomycesles yeast cells, more preferably 42.5% (w/w) or more protein per gram dry weight of such Saccharomycesales yeast cells (see FIG. 1). The Saccharomycesales yeast cells are preferably from the genus Cyberlindnera or Kluyveromyces or Wickerhamomyces, and more preferably the Saccharomycesles yeast cells are from the genus Cyberlindnera jadinii or Kluyveromyces lactis or Wickerhamomyces anomalus. Thus, Saccharomycesles yeast cells appear to produce more protein, when grown with ethanol as carbon source. Thus, such Saccharomycesales yeast cells appear to be suitable as single cell protein product, which is a source for, e.g., protein with the aim of at least partially, preferably fully replacing animal-derived protein sources, such as fish meal in animal feed.

Accordingly, when using such Saccharomycesles yeast cells as single cell protein product in animal feed, it was found that animal feed with up to 20% (w/w) yeast single cell protein product was well eaten by animals, particularly by aquatic species, such as fish or crustaceans. Moreover, when using such Saccharomycesles yeast cells as single cell protein product in animal feed, it was found that animal feed with up to 10% (w/w) yeast single cell protein product has a beneficial effect on the increase of the body weight of animals, particularly aquatic species, such as fish or crustaceans (see FIGS. 3 and 4). Also, animal feed comprising up to 20% (w/w) or 10% (w/w) yeast single cell protein product can advantageously fully replace animal-derived protein, such as fish meal (see, e.g., FIG. 2, particularly the columns “SCP1-10”, “SCP1-20”, “SCP2-10” and “SCP2-20”).

Interestingly, the present invention shows that animal feed comprising 10% (w/w) alternative protein sources, such as insect meal or single cell protein from methylotrophic bacteria using methane as carbon source, results in an inferior increase of body weight of animals in comparison to animal feed comprising 10% (w/w) single cell protein product according to the present invention (see FIG. 5).

In sum, there are at least five surprising findings of the present invention.

Saccharomycesles yeast cells grown with ethanol as carbon source produce give rise to a yeast single cell protein product with up to 42.5% (w/w) or more protein per gram dry weight of such Saccharomycesales yeast cells (see FIG. 1).

Animal feed comprising up to 20% (w/w) or 10% (w/w) yeast single cell protein product pursuant to the present invention can advantageously fully replace animal-derived protein, such as fish meal (see FIG. 2).

Animal feed comprising such yeast single cell protein product pursuant to the present invention up to 20% (w/w) is well eaten by animals, particularly aquatic species, such as fish or crustaceans.

Animal feed comprising such yeast single cell protein product pursuant to the present invention up to 10% (w/w) has a beneficial effect on the increase of the body weight of animals, particularly aquatic species, such as fish or crustaceans (see FIGS. 3 and 4).

Animal feed comprising up to 10% (w/w) yeast single cell protein product pursuant to the present invention may outperform animal feed also comprising up to 10% (w/w) alternative protein sources, such as insect meal or single cell protein product from methylotrophic bacteria using methane as carbon source (see FIG. 5).

In particular, the present invention relates to an animal feed comprising up to 20% (w/w) yeast single cell protein (SCP) product, wherein the yeast SCP product comprises Saccharomycesales yeast cells.

In the context of the present invention, the term “animal” refers to any animal except humans. Examples of animals are non-ruminants and ruminants. Ruminant animals include, for example, animals such as sheep, goats, cattle, e.g. beef cattle, cows, and young calves, deer, yank, camel, llama and kangaroo. Non-ruminant animals include monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); horses (including but not limited to hotbloods, coldbloods and warm bloods); crustaceans (including but not limited to shrimps and prawns); and fish including but not limited to warm water fish and cold water fish and thus, fish include but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, seabass, seabream, shiner, sleeper, snakehead, snapper, snook, sole, spinefoot, sturgeon, sunfish, sweetfish, tench, terror, tilapia, trout (preferably rainbow trout)), tuna, turbot, vendace, walleye and whitefish, with preferred warm water fish being selected from tilapia, seabream, seabass, or carp and/or preferred cold water fish being salmon or rainbow trout.

Of note, any animal referred to herein is preferably not a wildlife animal. Thus, said animal is preferably a farming animal and/or a livestock animal. In case of aquatic species, the animal is preferably an animal of an aquaculture. Herein, the term “aquaculture” relates to aqua-farming and thus, the farming of aquatic species such as fish or crustaceans in a variety of environments, including but not limited to tanks, lakes, ponds, or any other natural or man-made aquatic reservoirs that may be suitable for breeding, hatchery, rearing and harvesting of the aquatic species.

In the context of the present invention, the term “animal feed” (e.g., fish feed) refers to any compound, preparation, or mixture suitable for, or intended for intake by an animal (e.g., a fish). Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix). An animal feed additive (e.g., fish feed additive) is a formulated enzyme product which may further comprise e.g. vitamins, minerals, enzymes, amino acids, preservatives and/or antibiotics; i.e. a premix. The animal feed additive/premix is typically mixed in a feed mill with concentrates and/or forage such as vegetable protein, legumes or other plant material. Further, the animal feed is typically fed as a pelleted feed to mono-gastric animals.

In the context of the present invention, the term “single cell protein”, optionally abbreviated herein also as “SCP”, refers to a protein obtained by and/or derived from a (unicellular) microorganism. Thus, an SCP may refer to a protein purified and/or isolated from a microorganism's cell culture for example. Alternatively or additionally, SCPs may refer to microbial proteins are the dead dried cells of microorganisms. Hence, an “single cell protein product” or “SCP product” may or may not comprise one or more selected from the group of intact (unicellular) microorganism cells, disrupted (unicellular) microorganism cells, isolated proteins obtained by one or more (unicellular) microorganism(s), isolated proteins derived by one or more (unicellular) microorganism(s), purified proteins obtained by one or more (unicellular) microorganism(s), and purified proteins derived by one or more (unicellular) microorganism(s). While an (unicellular) microorganism may relate to a bacterium, a fungus like yeast and/or an algae, said (unicellular) microorganism is yeast according to the present invention. SCP products from yeast offer the advantage of providing comparatively high protein contents, while at the same time said products can be produced on industrial scale at comparatively low cost, independent from seasonal effects and with comparatively low harvesting efforts. Thus, yeast SCP products are highly advantageous.

In the context of the present invention, the term “yeast” refers to a eukaryotic, unicellular microorganism classified as a member of the fungus kingdom that mostly reproduce asexually by mitosis with asymmetric division processes also being known as budding. Further herein, said term preferably relates to yeast cells, which can be grown under artificial and/or lab conditions, e.g. as in vitro culture conditions, and in particular under standard laboratory conditions. Said term preferably also embraces yeast cells of a single type that have been grown in the laboratory for several generations and thus, said term preferably embraces also potential mutants of a yeast cell and/or strain. Herein, yeast is preferably Saccharomycesales yeast.

A “yeast cell” is a cell of a yeast, preferably a cell of a yeast as described herein.

In the context of the present invention, the term “Saccharomycesles” refers to the order Saccharomycesles within the phylum Ascomycota. Members of Saccharomycesles are also known and sometimes referred to as budding yeasts.

In the context of the present invention, the term “w/w” is intended to be understood as “weight by weight” and thus refers to the proportion of a particular substance within a mixture, as measured by weight or mass.

According to the present invention, the animal feed comprises up to 20% (w/w) yeast SCP product. More specifically, the animal feed may comprise up to 20% (w/w), up to 19% (w/w), up to 18% (w/w), up to 17% (w/w), up to 16% (w/w), up to 15% (w/w), up to 14% (w/w), up to 13% (w/w), up to 12% (w/w), up to 11% (w/w), up to 10% (w/w), up to 9% (w/w), up to 8% (w/w), up to 7% (w/w), up to 6% (w/w), or up to 5% (w/w) yeast SCP product. Preferably, the animal feed comprises up to 15% (w/w) or up to 10% (w/w) yeast SCP product, more preferably up to 10% (w/w) yeast SCP.

Preferably, the animal feed comprises up to 10% (w/w) yeast SCP product, wherein the yeast SCP product comprises Saccharomycesles yeast cells. This is especially advantageous to increase weight of an animal that is fed with said animal feed like shrimps or a fish such as rainbow trout. This is exemplified herein, for example in FIGS. 3 and 4.

It is further preferred that the animal feed according to the present invention comprises up to 20% (w/w) or up to 10% (w/w) yeast SCP product, wherein the yeast SCP product comprises Saccharomycesales yeast cells, and wherein said SCP product comprises ethanol fed Saccharomycesles yeast cells. Thus, the SCP product comprises preferably ethanol fed Saccharomycesles yeast cells. This is advantageous as the use of ethanol as carbon source paves the way for utilizing alternative SCP product production sources like waste. Thus, the environmental carbon footprint of the animal feed production according to the present invention can be minimized compared to current SCP production approaches and a sustainable SCP protein production envisioned.

Accordingly, it is also preferred that the animal feed according to the present invention comprises up to 20% (w/w), or preferably up to 10% (w/w), yeast SCP product, wherein the yeast SCP product comprises Saccharomycesales yeast cells, and wherein said Saccharomycesles yeast cells are capable of producing with ethanol as carbon source 34% (w/w) or more, preferably 41% (w/w) or more, more preferably 42.5% (w/w) or more protein per gram dry weight of said Saccharomycesles yeast cells. Such high protein contents of yeast SCPs are advantageous for using the animal feed for feeding animals and especially for increasing weight of animals fed therewith.

SCP product producer may vary in their ability to use and/or utilize ethanol as carbon source for SCP production. Thus, the yeast SCP product preferably comprises Saccharomycesles yeast cells, wherein said Saccharomycesles yeast cells are Saccharomycesales yeast cells from one or more Saccharomycesles yeast genera, species and/or strains that are capable of using ethanol as carbon source. For example, the Saccharomycesles yeast cells may be Saccharomycesles yeast cells from one or more genera selected from the group consisting of Cyberlindnera, Kluyveromyces, Wickerhamomyces, Yarrowia and Pichia.

More specifically, the yeast SCP product comprises Saccharomycesales yeast cells, wherein said Saccharomycesales yeast cells are preferably Saccharomycesales yeast cells selected from the group consisting of Pichia anomala, Yarrowia lipolytica, Wickerhamomyces anomalus, Cyberlindnera jadinii and/or Kluyveromyces lactis.

Preferably, the Saccharomycesales yeast cells are not Saccharomyces cerevisiae yeast cells. Thus, the animal feed may comprises yeast SCP product, wherein the yeast SCP product does not comprise Saccharomyces cerevisiae yeast cells. Thus, the SCP product may be free of intact and/or disrupted Saccharomyces cerevisiae yeast cells. It may even be envisioned that the animal feed may not comprise Saccharomyces cerevisiae yeast cells.

Thus, it is particularly preferred that the animal feed according to the present invention comprises up to 20% (w/w) or up to 10% (w/w) yeast SCP product, wherein the yeast SCP product comprises Saccharomycesles yeast cells, and wherein said Saccharomycesles yeast cells are yeast cells from the genus Wickerhamomyces, Cyberlindnera, Kluyveromyces, Yarrowia and/or Pichia, preferably from Cyberlindnera, Kluyveromyces and/or Wickerhamomyces. This is advantageous as yeast cells from said genera are capable of growing on a culture medium comprising ethanol as carbon source as also shown herein in the Examples (see, e.g., FIG. 1).

Preferably, that the animal feed according to the present invention comprises up to 20% (w/w) or up to 10% (w/w) yeast SCP product, wherein the yeast SCP product comprises Saccharomycesales yeast cells, and wherein said Saccharomycesles yeast cells are preferably from Cyberlindnera jadinii, Kluyveromyces lactis, Wickerhamomyces anomalus, Pichia anomala and/or Yarrowia lipolytica, more preferably from Wickerhamomyces anomalus, Cyberlindnera jadinii and/or Kluyveromyces lactis. This is especially advantageous as SCP products from said species can have, e.g., 34% (w/w) or more, preferably 41% (w/w) or more, more preferably 42.5% (w/w) or more, protein per dry matter (see FIG. l ) and moreover, can fully replace animal-derived protein, such as fish meal, in an animal feed according to the present invention (see, e.g., FIG. 2, particularly the columns “SCP1-10”, “SCP1-20”, “SCP2-10” and “SCP2-20”).

Preferably, that the animal feed according to the present invention comprises up to 20% (w/w) or up to 10% (w/w) yeast SCP product, wherein the yeast SCP product comprises Saccharomycesles yeast cells, and wherein said Saccharomycesles yeast cells are derived and/or are from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950, Kluyveromyces lactis CBS 2896, Wickerhamomyces anomalus CBS 2576 and/or Yarrowia lipolytica CBS 7504, preferably from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950 and/or Kluyveromyces lactis CBS 2896. This is advantageous as an animal feed with up to 20% (w/w) or up to 10% (w/w) SCP product from said yeast strains can be used as an animal feed and moreover, can have a positive impact on the body weight of an animal that is fed therewith as also shown in Example 1 herein (see, e.g., FIGS. 3 and 4).

In the context of the present invention, the term “derived from” preferably refers to yeast cells, which were originally obtained from a given yeast strain and thus originate from said given yeast strain. Such derived cells may differ from said given yeast strain due to naturally occurring and/or artificially introduced alterations like genetic mutations, but preferably have similar characteristics as cells from the yeast strain they originated from. Such similar characteristics are preferably the capability to produce with ethanol as carbon source 34% (w/w) or more protein per gram dry weight of yeast cells, preferably 41% (w/w) or more protein per gram dry weight of yeast cells, more preferably 42.5% (w/w) or more protein per gram dry weight of yeast cells. A skilled person can readily test such a capability by culturing yeast cells with ethanol as carbon source, whereby a range of ethanol concentrations as carbon source are tested. Accordingly, cells that are derived from a given strain may have, preferably on genome level, a sequence identity of 80% or more, preferably of 85% or more, more preferably of 90% or more, even more preferably of 95% or more to the respective strain that can be seen as reference. Thus, a derived cell may have a sequence identity of at least, e.g., 90%, 91%, 92%, 93%, 94%, 95%, 96%, 97%, 98% or 99% to the respective reference, preferably on genome level.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of nucleotide and/or amino acids with a respective sequence in question—with respect to the number of residues in the longer of these two sequences. Sequence identity is measured by dividing the number of identical nucleotides and amino acid residues, respectively, by the total number thereof and multiplying the product by 100. A skilled artisan will recognize available computer programs, for example BLAST (Altschul et al., 1997), BLAST2 (Altschul et al., 1990), FASTA (Pearson and Lipman, 1988), GAP (Needleman and Wunsch, 1970), Smith-Waterman (Smith and Waterman, 1981), and Wisconsin GCG Package, for determining sequence identity using standard parameters. The percentage of sequence identity can, for example, be determined herein using the program BLASTP, version 2.2.5, Nov. 16, 2002 (Altschul et al., 1997), calculating the percentage of numbers of “positives” (homologous amino acids) from the total number of amino acids selected for the alignment.

Accordingly, “percent (%) sequence identity” with respect to cells and/or strains described herein is preferably defined on nucleic acid level and thus, as the percentage of nucleotides in a candidate sequence that are identical with the nucleotides in the reference sequence, after aligning the sequences and introducing gaps, if necessary, to achieve the maximum percent sequence identity, and not considering any conservative substitutions as part of the sequence identity. Alignment for purposes of determining percent nucleotides sequence identity can be achieved in various ways that are within the skill in the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same is applicable to amino acid sequences, mutatis mutandis.

For example, an appropriate alignment for nucleic acid sequences is provided by the local homology algorithm of Smith and Waterman, (1981), Advances in Applied Mathematics 2:482-489. This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986), Nucl. Acids Res. 14 (6): 6745-6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters.

In the context of the present invention, the following illustrative example may be considered. When an obtained yeast strain, which may have been an officially deposited yeast strain initially, has been cultivated and propagated for some generations, e.g. under laboratory conditions, the resulting yeast cells may be genetically identically to the deposited genetic material of the initial yeast strain. In said case, the cells “are” the cells from the deposited strain and have 100% sequence identity with the deposited material on genome level. However, a portion of the cells or even all cells may show some degree of genetic and/or epigenetic variation, for example due to one or more mutations. In said case, the cells under study “are derived” from the initial strain the genetic material and/or cells of which is deposited. Such mutations may be naturally occurring during cultivation and propagation of cells. Alternatively or additionally, though preferred, such mutations may be artificially introduced, e.g., by genetic engineering. Consequently, derived cells may potentially exhibit, e.g., a variation in the protein per dry matter (%) (w/w), the amount of essential amino acids and/or composition of essential amino acids, compared to the initial yeast strain the cells are derived from. The obtained yeast cells may also be referred to as mutants compared to the cells and/or strain they are derived from.

Herein, the term “genetic engineering” is used in its broadest sense for methods known to the person skilled in the art to modify desired nucleic acids in vitro and in vivo, e.g. by targeted mutagenesis and/or recombinant DNA technology. Accordingly, said methods may comprise cloning, sequencing and transformation of recombinant nucleic acids, and appropriate vectors, primers, enzymes, host cells and the like are known by the skilled artisan. Preferably, genetically engineered cells are genetically engineered in view of high protein per dry matter (%), suitable essential amino acid composition, efficient ethanol usage as carbon source and the like in the context of the present invention.

Furthermore, herein the terms “mutated” and “mutant” mean permanent (epi-) genetic modification(s) of genetic material, i. e. nucleic acids, caused, for example, naturally or by physical means or chemical compounds/substances/agents such as EMS. Said modifications include point mutations, transitions, transversions, deletion/insertion/addition of one or more bases within a nucleic acid/gene/chromosome thereby optionally modifying the nucleic acid/gene/chromosome which can cause, inter alia, phenotypic effects like varying protein per dry matter (%) (w/w). Furthermore, such modification(s) may be induced by methods known to the person skilled in the art. The skilled person is also aware of suitable methods to select cells in view of one or more favorable and/or desired phenotypic trait(s) like an increase of protein per dry matter (%) (w/w) and/or utilization of ethanol as carbon source.

Preferably, the Saccharomycesales yeast cells are not genetically engineered. Thus, the animal feed according to the present invention comprises up to 20% (w/w) or up to 10% (w/w) yeast SCP product, wherein the yeast SCP product comprises Saccharomycesles yeast cells, wherein said Saccharomycesles yeast cells are preferably not genetically engineered. This is advantageous to ensure that the SCP product comprised in the animal feed comprises only well-defined and/or well characterized yeast cells but no mutated an thus, potentially undefined and/or uncharacterized yeast cells. This is also advantageous for a constant SCP product quality.

As regards the SCP product comprised in the animal feed according to the present invention it is preferred that said yeast SCP product comprises dried Saccharomycesles yeast cells. Thus, the animal feed according to the present invention comprises up to 20% (w/w), or preferably up to 10% (w/w), yeast SCP product, wherein said SCP product comprises Saccharomycesles yeast cells, wherein said Saccharomycesles yeast cells are preferably at least partially dried. It is furthermore preferred that said dried Saccharomycesles yeast cells are intact or disrupted or a mixture of intact and disrupted cells. Thus, the animal feed of the present invention comprises up to 20% (w/w), or preferably up to 10% (w/w), yeast SCP product, wherein said SCP product comprises preferably dried Saccharomycesles yeast cells, wherein said dried Saccharomycesles yeast cells may be intact and/or disrupted (cells).

Additionally or alternatively, it is preferred that the yeast SCP product comprised in the animal feed according to the present invention comprises all essential amino acids. This is advantageous, as an animal feed comprising a yeast SCP product comprising all essential amino acids is capable of fully replacing currently used animal-and/or plant-derived protein sources in animal feed. Thus, said SCP product can represent an alternative protein source that does not require any supplementation in view of essential amino acids.

In the context of the present invention, the term “essential amino acid” preferably refers to amino acids that cannot be synthesized by an animal from metabolic intermediates. Thus, such amino acids have to be supplied from an exogenous diet as they are required, e.g., for growth. Although variations may be possible, e.g., depending on the metabolic state of an animal, in general the following nine amino acids are considered essential: phenylalanine, valine, tryptophan, threonine, isoleucine, methionine, histidine, leucine, and lysine. Notably, in terms of nutrition, said nine essential amino acids are obtainable by a single complete protein containing all the essential amino acids. Such complete proteins can be derived from animal-based sources of nutrition, whereas plant-based foods represent commonly a source for essential amino acids in the form of incomplete proteins.

As regards the SCP product comprised in the animal feed according to the present invention, it is preferred that said yeast SCP product comprises 34% (w/w) or more protein per gram dry weight of Saccharomycesles yeast cells, preferably 41% (w/w) or more protein per gram dry weight of Saccharomycesles yeast cells, more preferably 42.5% (w/w) or more protein per gram dry weight of Saccharomycesles yeast cells. Such protein contents of yeast SCPs are advantageous for using the animal feed for feeding an animal, and in particular for increasing body weight of an animal fed therewith.

Preferably, the animal feed according to the present invention further comprises less than 25% (w/w) plant-based protein products. This has the advantage that the percentage of plant-based protein products can be decreased compared to currently used animal feed formulations as shown illustratively in the Examples herein. Thus, plant-derived proteins that tend to be poorly converted into animal-derived protein in traditional farming can be at least partially replaced by SCP products that may be better converted, while at the same time are less associated with potential ethical conflicts between food and feed production. Thus, compared to conventional plant-derived protein production less greenhouse gases may be produced, making the animal feed to a more sustainable alternative.

Additionally or alternatively, the animal product according to the present invention preferably further comprises 5% (w/w) or less fish meal, preferably the animal feed comprises no fish meal. This is in particular advantageous for reducing or even overcoming catching of wild fish stocks for fish meal production. Thus, loss of biodiversity can be reduced and the animal feed according to the present invention represents furthermore a comparatively sustainable alternative to several of the currently available animal feeding solutions comprising fish meal.

Preferably, the animal feed according to the present invention comprises from 1 to 25% (w/w) oil. Thus, the animal feed may comprise for example about 5% (w/w), about 10% (w/w), about 15% (w/w), or about 20% (w/w) oil. Also, the animal feed may comprise for example from 5% (w/w) to 10% (w/w), from 5% (w/w) to 20% (w/w) or from 10% (w/w) to 15% (w/w) or from 15% (w/w) to 20% (w/w) oil. Preferably, the animal feed comprises 1% (w/w) to 15% (w/w), more preferably from 5% (w/w) to 10% (w/w) oil.

The animal feed according to the present invention is preferably a feed for pigs, cows, or sheep. Alternatively or additionally, said animal feed is preferably a feed for aquatic species. In case of the animal feed being a feed for aquatic species said species are preferably selected from crustaceans or fish. Thus, the animal feed may be a feed for crustaceans and/or fish. When the animal feed is a feed for crustaceans, said crustaceans are preferably shrimps. Additionally or alternatively, in case the animal feed is a feed for fish, said fish may preferably be warm water fish or cold water fish. When said fish are warm water fish, said fish are preferably selected from the group consisting of tilapia, seabream, seabass, and carp. Preferably, said fish are cold water fish with said fish being selected from salmon or rainbow trout. It is particularly preferred that the animal feed according to the present invention is a feed for shrimps, salmon and/or rainbow trout.

The present invention relates also to the use of the animal feed as disclosed herein for feeding an animal. For example, the animal feed comprising up to 20% (w/w), preferably up to 20% (w/w), yeast SCP product as disclosed herein above, wherein the yeast SCP product comprises preferably Saccharomycesles yeast cells derived from Wickerhamomyces anomalus IFO 569, Wickerhamomyces anomalus CBS 1980, Cyberlindnera jadinii FERM-BP1656, Cyberlindnera jadinii ATCC 26387, Cyberlindnera jadinii ATCC 9950 and/or Kluyveromyces lactis CBS 2896, the animal feed preferably further comprising 5% (w/w) or less fish meal and 20% (w/w) or less plant-based protein products, may be used for feeding animals. This is especially advantageous as such animal feed is well eaten by animals, particularly by aquatic species, such as fish or crustaceans.

The present invention relates also to the use of the animal feed as disclosed herein for increasing body weight of an animal. For example, the animal feed comprising up to 20% (w/w), preferably up to 20% (w/w), yeast SCP product as disclosed herein above, wherein the yeast SCP product comprises preferably Saccharomycesales yeast cells derived from Wickerhamomyces anomalus IFO 569, Wickerhamomyces anomalus CBS 1980, Cyberlindnera jadinii FERM-BP1656, Cyberlindnera jadinii ATCC 26387, Cyberlindnera jadinii ATCC 9950 and/or Kluyveromyces lactis CBS 2896, the animal feed preferably further comprising 5% (w/w) or less fish meal and 20% (w/w) or less plant-based protein products, may be used for feeding animals. This is especially advantageous for increasing the body weight of animals like animals of aquatic species, such as fish or crustaceans.

Moreover, either of the two uses according to the present invention and thus, the use of the inventive animal feed disclosed for feeding an animal and/or for increasing body weight of an animal, has the advantage that animals can be fed with the inventive animal feed comprising up to 20% (w/w) or 10% (w/w) yeast SCP product without negative effect on eating behavior, amount eaten, and/or body weight. Thus, the inventive animal feed can advantageously be used to partially, preferably even fully replace animal-derived protein, such as fish meal, in animal feed with its use in animal feeding being associated with well eating and/or weight increase of the animals. Moreover, use of the inventive animal feed, in particular animal feed comprising up to 10% (w/w) yeast SCP product, for feeding an animal and for increasing weight of an animal may even result in better eating and weight increase, respectively, compared to the use of an animal feed comprising up to 10% (w/w) alternative protein sources, such as insect meal or single cell protein product from methylotrophic bacteria using methane as carbon source.

The animal according to any one of the two uses according to the present invention is preferably a pig, cow, or sheep. Alternatively, said animal is preferably an aquatic species and thus, an animal of an aquatic species. When the animal is an animal of an aquatic species, the aquatic species is preferably selected from crustaceans, preferably shrimps, or fish. Further, when the animal is a fish, said fish is preferably a warm water fish or a cold water fish, wherein said warm water fish is preferably a warm water fish selected from the group consisting of tilapia, seabream, seabass, and carp, and wherein said cold water fish is preferably a salmon or a rainbow trout.

Of note, it is preferred that the uses of the present invention are non-therapeutic. Indeed, the improvement of the weight of an animal like of aquatic species is deemed to be associated and/or to contribute to, e.g. health or growth of the animal, which is by way of common sense non-therapeutic.

Accordingly, it is preferred in the context of the uses of the present invention that the animal is healthy.

It is to be noted that in case of any definition given herein, the respective definition of a term, phrase, and/or abbreviation applies vice versa throughout the specification. Furthermore, all definition given herein are intended to encompass all grammatical forms.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art and that such modifications and variations are considered to be within the scope of this disclosure.

### V. BRIEF DESCRIPTION OF THE FIGURES

FIG. 1 shows yeast single cell protein product from different yeast strains when grown with ethanol as carbon source. Shown is the protein on dry matter (%) (w/w) of obtained SCP product (y-axis) per yeast strain (x-axis) of in total 8 genera.

FIG. 2 shows the animal feed formulation used in a 12 week feeding trial of rainbow trout. SCP1 stands for single cell protein product from Cyberlindnera jadinii. SCP2 stands for single cell protein product from Kluyveromyces lactis. SCP1-5 stands an animal feed comprising 5% (w/w) single cell protein product from Cyberlindnera jadinii; SCP1-10 stands an animal feed comprising 10% (w/w) single cell protein product from Cyberlindnera jadinii; SCP1-20 stands an animal feed comprising 20% (w/w) single cell protein product from Cyberlindnera jadinii. SCP2-5 stands an animal feed comprising 5% (w/w) single cell protein product from Kluyveromyces lactis; SCP2-10 stands an animal feed comprising 10% (w/w) single cell protein product from Kluyveromyces lactis; SCP2-20 stands an animal feed comprising 20% (w/w) single cell protein product from Kluyveromyces lactis.

FIG. 3 shows the final body weight of rainbow trout at the end of the 12 week feeding trial. SCP1 5% stands an animal feed comprising 5% (w/w) single cell protein product from Cyberlindnera jadinii; SCP1 10% stands an animal feed comprising 10% (w/w) single cell protein product from Cyberlindnera jadinii; SCP1 20% stands an animal feed comprising 20% (w/w) single cell protein product from Cyberlindnera jadinii. SCP2 5% stands an animal feed comprising 5% (w/w) single cell protein product from Kluyveromyces lactis;

SCP2 10% stands an animal feed comprising 10% (w/w) single cell protein product from Kluyveromyces lactis; SCP2 20% stands an animal feed comprising 20% (w/w) single cell protein product from Kluyveromyces lactis.

FIG. 4 shows the increase of the final body weight of rainbow trout at the end of the 12 week feeding trial in relation to control (animal feed not containing single cell protein product). SCP1 5% stands an animal feed comprising 5% (w/w) single cell protein product from Cyberlindnera jadinii; SCP1 10% stands an animal feed comprising 10% (w/w) single cell protein product from Cyberlindnera jadinii; SCP1 20% stands an animal feed comprising 20% (w/w) single cell protein product from Cyberlindnera jadinii. SCP2 5% stands an animal feed comprising 5% (w/w) single cell protein product from Kluyveromyces lactis; SCP2 10% stands an animal feed comprising 10% (w/w) single cell protein product from Kluyveromyces lactis; SCP2 20% stands an animal feed comprising 20% (w/w) single cell protein product from Kluyveromyces lactis.

FIG. 5 shows the increase of the final body weight of rainbow trout at the end of the 12 week feeding trial in relation to control (animal feed not containing single cell protein product) and in relation to animal feed containing alternative protein sources, e.g. insect meal or a single cell protein product obtained by using methane as substrate. SCP1 5% stands an animal feed comprising 5% (w/w) single cell protein product from Cyberlindnera jadinii; SCP1 10% stands an animal feed comprising 10% (w/w) single cell protein product from Cyberlindnera jadinii; SCP1 20% stands an animal feed comprising 20% (w/w) single cell protein product from Cyberlindnera jadinii. SCP2 5% stands an animal feed comprising 5% (w/w) single cell protein product from Kluyveromyces lactis; SCP2 10% stands an animal feed comprising 10% (w/w) single cell protein product from Kluyveromyces lactis; SCP2 20% stands an animal feed comprising 20% (w/w) single cell protein product from Kluyveromyces lactis. “IM10” stands for animal feed containing 10% (w/w) insect meal; “IM20” stands for animal feed containing 20% (w/w) insect meal. “KP5” stands for animal feed containing 5% (w/w) single cell protein product obtained by using methane as substrate; “KP10” stands for animal feed containing 10% (w/w) single cell protein product obtained by using methane as substrate; “KP20” stands for animal feed containing 20% (w/w) single cell protein product obtained by using methane as substrate. “additive” stands for Balancius, a product launched for broilers to improve intestinal nutrient absorption. “IM10” and “IM20” in the middle part of the Figure stand for animal feed containing 10% (w/w) and 20% (w/w) insect meal, respectively, and in the right part of the Figure for animal feed containing 10% (w/w) and 20% (w/w) insect protein meal, respectively. In all trials tested insect meal was Protein x from Protix.

### VI. EXAMPLES

### Example 1: SCP Product Generation

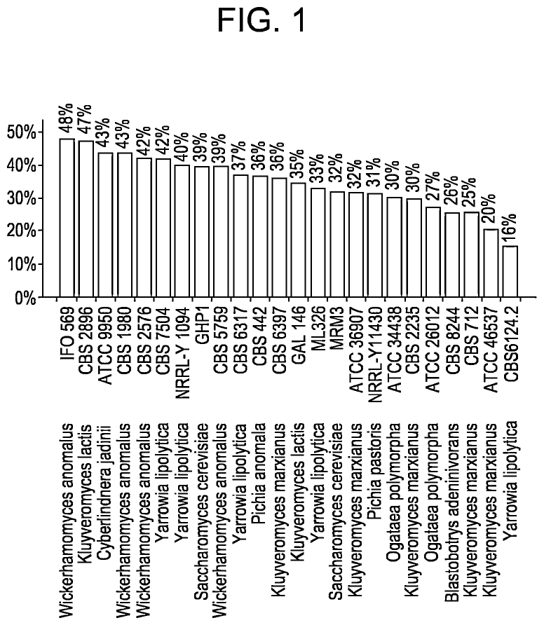

Single cell protein products were generated using Saccharomycesales yeast cells from different yeast genera using ethanol as carbon source. More specifically, strains of the genera Cyberlindnera, Kluyveromyces, Wickerhamomyces, Yarrowia, Saccharomyces, Pichia, Ogateae and Blastobotrys were investigated. Protein (g) per dry matter (%) (w/w) of SCP product obtained in the Example is given in FIG. 1 for SCP products obtained using Saccharomycesles yeast cells from Yarrowia lipolytica, Kluyveromyces marxianus, Blastobotrys adeninivorans, Ogateae polymorpha, Pichia anomala, Pichia pastorsi, Saccharomyces cerevisiae, Wickerhamomyces anomalus, Cyberlindnera jadinii and Kluyveromyces lactis. As shown, comparatively well performing yeast cells were Saccharomycesales yeast cells from Wickerhamomyces anomalus, Kluyveromyces lactis, and Cyberlindnera jadinii in view of protein (g) per dry matter (g) given as protein on dry matter (%) with (up to) over 41% (w/w) observed.

### Example 2: Rainbow Trout Feeding Trial

### Study Design

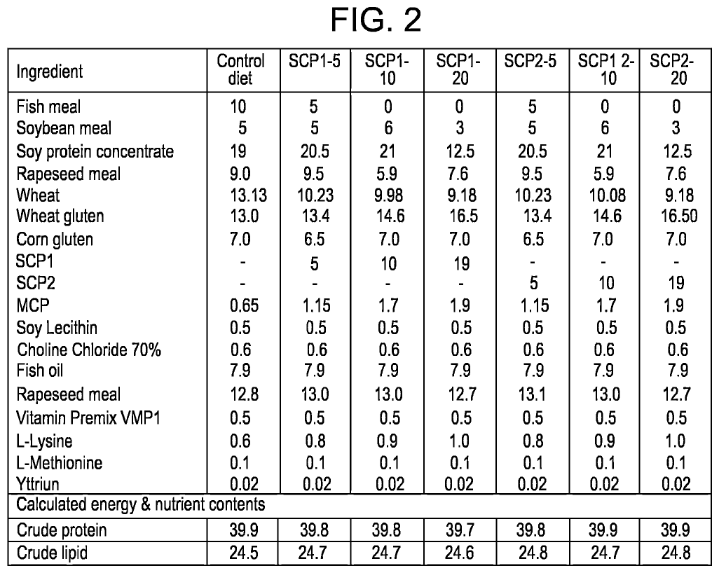

A control diet containing 10% (w/w) of fish meal (FM) and 20% (w/w) soy protein concentrate (SPC) was used. Further, two test products (SCP1 and SCP2) were investigated. Said test products were based on the control diet, wherein fish meal was partially replaced (5% diets), completely replaced (10% diets), or completely replaced together with part of the soy protein concentrate (20% diets). Replacement was done using single cell protein products from yeast, namely from Cyberlindnera jadinii FERM-BP1656 (SCP1) or from Kluyveromyces lactis CBS 2896 (SCP2). Diets were isonitrogenous and isolipidic. Details of the formulation per diet are given in FIG. 2.

Diets were fed to triplicate groups of rainbow trout with an initial IBW of 50g for 12 weeks. Thus, the feeding trial length was 12 weeks. Further, the feeding trial comprised three inclusion levels, namely 5%, 10% and 20% as explained, for each of the two test products. Thus, in total 7 groups were investigated, namely a control, SCP1 5%, SCP1 10%, SCP 20%, SCP2 5%, SCP2 10%, and SCP2 20%. As all groups were investigated with three replicates each, in total 21 replicates investigated, corresponding to in total 21 fish tanks.

Herein, fish tanks had a volume of 250L and comprised 20 fish per tank. Temperature was set to 15° C. Animals were manually fed twice daily. Weighting was done bi-weekly in the form of bulk weighting.

### Results

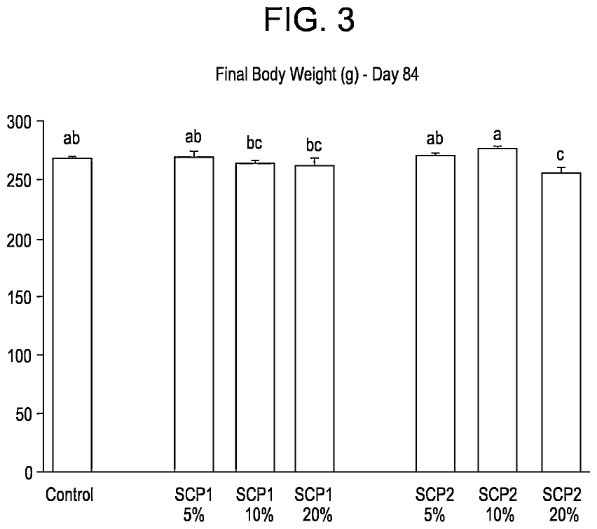

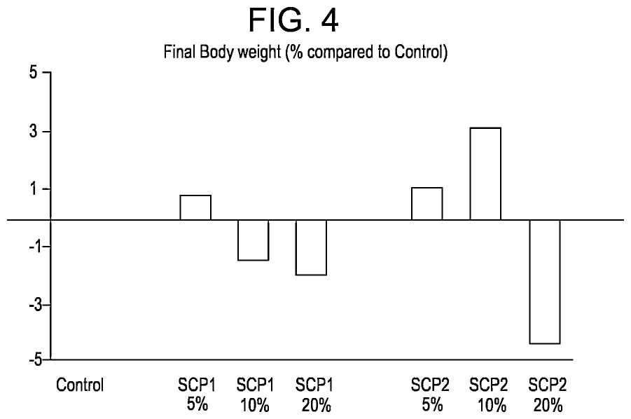

Final body weight was similar for 6 out of the 7 groups. More specifically, the two new sustainable protein sources showed comparable growth performance when included up to 10% in the feed ration when compared to a marine control diet as it can be seen in FIGS. 3 and 4; only SCP2 20% showed a significant decrease in the final body weight (4%). Furthermore, feeding with SCP1 resulted in +0.81 (5%) to-2.01% (20%) final body weight increase when compared to control, and feeding with SCP2 resulted in +3.13 (10%) to −4.4% (20%) final body weight increase when compared to control, as shown in FIGS. 3 and 4. It is to be noted that animals were eating well in said SCP2 20% group.

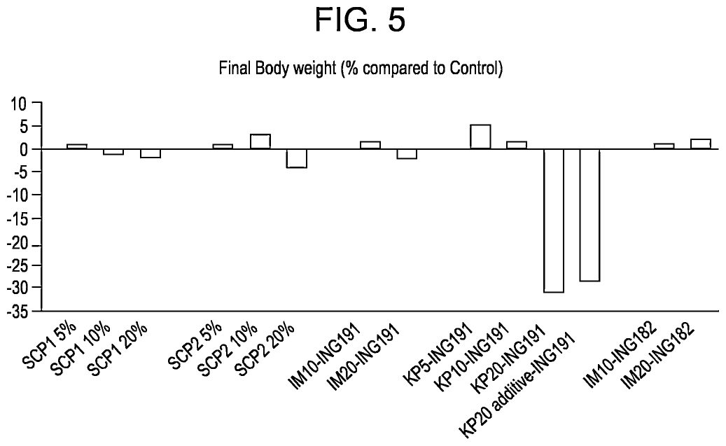

Final body weight was also compared to further feeding diets. In particular, FIG. 5 shows a comparison of the percentage (%) of final body weight compared to control using the SCP diets under study as well as with alternative protein replacements (IM=insect meal (middle Figure part) and insect protein meal (right Figure part), respectively, tested insect meal was Protein x from Protix; KP=knip bio: a single cell protein obtained using methane as substrate; “additive”=Balancius). As it can be seen in FIG. 5, effect of 20% inclusion of the KP ingredient massively impacted the final body weight that was found to be reduced by approximately 25%.

### Experimental Summary

Data showed normally good results with SCP replacement ingredients up to 10%. Inclusions (diets) above that level generally impact growth in a significant manner (either related to decreased palatability of the diets that include high levels of alternative ingredients; either because the ingredients have anti-nutritional factors that might impair digestion and absorption of the nutrients)

Thus, the use of two different alternative sources obtained from yeast (C. jadinii and K. lactis) showed equal or improved growth at inclusion levels up to 10% in the diet of rainbow trout. The inclusion of 20% of the ingredients slightly decreased the performance of the animals after 12 weeks of feeding. Thus, the performed experiment showed that new protein sources can effectively replace fish meal and soy protein concentrate up to 20% in practical aquaculture diets for freshwater species like rainbow trout.

The present invention may also be summarized in the following items:
1. An animal feed comprising up to 20% (w/w) yeast single cell protein product, wherein the yeast single cell protein product comprises Saccharomycesales yeast cells.
2. The animal feed of item 1 comprising up to 10% (w/w) yeast single cell protein product, wherein the yeast single cell protein product comprises Saccharomycesales yeast cells.
3. The animal feed of item 1 or 2, wherein the yeast single cell protein product comprises Saccharomycesles yeast cells that are not Saccharomyces cerevisiae yeast cells.
4. The animal feed of any one of the preceding items, wherein the yeast single cell protein product does not comprise Saccharomyces cerevisiae yeast cells.
5. The animal feed of any one of the preceding items, wherein said single cell protein product comprises ethanol fed Saccharomycesles yeast cells.
6. The animal feed of any one of the preceding items, the Saccharomycesales yeast cells are yeast cells from the genus Cyberlindnera, Kluyveromyces, Wickerhamomyces, Pichia or Yarrowia, preferably from the genus Cyberlindnera or Kluyveromyces or Wickerhamomyces.
7. The animal feed of any one of the preceding items, the Saccharomycesales yeast cells are from Cyberlindnera jadinii, Kluyveromyces lactis, Wickerhamomyces anomalus, Pichia anomala or Yarrowia lipolytica, preferably from Cyberlindnera jadinii or Kluyveromyces lactis or Wickerhamomyces anomalus.
8. The animal feed of any one of the preceding items, wherein the Saccharomycesles yeast cells are derived from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950, Kluyveromyces lactis CBS 2896, Wickerhamomyces anomalus CBS 2576 or Yarrowia lipolytica CBS 7504, preferably from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950 or Kluyveromyces lactis CBS 2896.
9. The animal feed of any one of the preceding items, wherein the Saccharomycesles yeast cells are from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950, Kluyveromyces lactis CBS 2896, Wickerhamomyces anomalus CBS 2576 or Yarrowia lipolytica CBS 7504, preferably from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950 or Kluyveromyces lactis CBS 2896.
10. The animal feed of any one of the preceding items, wherein said Saccharomycesles yeast cells are capable of producing with ethanol as carbon source 34% (w/w) or more protein per gram dry weight of said Saccharomycesles yeast cells, preferably 41% (w/w) or more protein per gram dry weight of said Saccharomycesles yeast cells, more preferably 42.5% (w/w) or more protein per gram dry weight of said Saccharomycesles yeast cells.
11. The animal feed of any one of the preceding items, wherein said yeast single cell protein product comprises all essential amino acids.
12. The animal feed of any one of the preceding items, wherein said Saccharomycesles yeast cells are not genetically engineered.
13. The animal feed of any one of the preceding items, wherein said yeast single cell protein product comprises 34% (w/w) or more protein per gram dry weight of Saccharomycesles yeast cells, preferably 41% (w/w) or more protein per gram dry weight of Saccharomycesles yeast cells, more preferably 42.5% (w/w) or more protein per gram dry weight of Saccharomycesles yeast cells.
14. The animal feed of any one of the preceding items, wherein said yeast single cell protein product comprises dried Saccharomycesles yeast cells.
15. The animal feed of item 14, wherein said dried Saccharomycesales yeast cells are intact or disrupted or a mixture of intact and disrupted cells.
16. The animal feed of any one of the preceding items, wherein said animal feed further comprises less than 25% (w/w) plant-based protein products.
17. The animal feed of any one of the preceding items, wherein said animal feed further comprises 5% (w/w) or less fish meal.
18. The animal feed of any one of the preceding items, wherein said animal feed comprises no fish meal.
19. The animal feed of any one of the preceding items, wherein said animal feed comprises from 1 to 25% (w/w) oil.
20. The animal feed of any one of the preceding items, wherein said animal feed is a feed for pigs, cows, or sheep.
21. The animal feed of any one of the preceding items, wherein said animal feed is a feed for aquatic species.
22. The animal feed of item 21, wherein said aquatic species are selected from crustaceans or fish.
23. The animal feed of item 22, wherein said fish are warm water fish or cold water fish.
24. The animal feed of item 23, wherein said warm water fish are selected from tilapia, seabream, seabass, or carp.
25. The animal feed of item 23, wherein said cold water fish are selected from salmon or rainbow trout.
26. The animal feed of item 22, wherein said crustaceans are shrimps.
27. Use of the animal feed of any one of the preceding items for feeding an animal.
28. Use of the animal feed of any one of items 1 to 26 for increasing body weight of an animal.
29. The use of item 27 or 28, wherein said animal are aquatic species.
30. The use of item 29, wherein said aquatic species are selected from crustaceans or fish.
31. The use of item 30, wherein said fish are warm water fish or cold water fish.
32. The use of item 31, wherein said warm water fish are selected from tilapia, seabream, seabass, or carp.
33. The use of item 32, wherein said cold water fish are selected from salmon or rainbow trout.
34. The use of item 30, wherein said crustaceans are shrimps.
35. The use of item 27 or 28, wherein said animal is a pig, cow, or sheep.

Those skilled in the art will recognize, or be able to ascertain, using not more than routine experimentation, many equivalents to the specific embodiments of the present invention described herein. Such equivalents are intended to be encompassed by the present invention.

Unless otherwise stated, the following terms used in this document, including the description and claims, have the definitions given below.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the present invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Other embodiments are within the following claims. In addition, where features or aspects of the present invention are described in terms of Markush groups, those skilled in the art will recognize that the present invention is also thereby described in terms of any individual member or subgroup of members of the Markush group.

All publications cited throughout the text of this specification (including all patents, patent applications, scientific publications, manufacturer's specifications, instructions, etc.) are hereby incorporated by reference in their entirety. Nothing herein is to be construed as an admission that the present invention is not entitled to antedate such disclosure by virtue of prior invention. To the extent the material incorporated by reference contradicts or is inconsistent with this specification, the specification will supersede any such material.

1. An animal feed comprising up to 20% (w/w) yeast single cell protein product, wherein the yeast single cell protein product comprises Saccharomycesales yeast cells.

2. The animal feed of claim 1 comprising up to 10% (w/w) yeast single cell protein product, wherein the yeast single cell protein product comprises Saccharomycesales yeast cells.

3. The animal feed of claim 1, wherein the yeast single cell protein product comprises Saccharomycesales yeast cells that are not Saccharomyces cerevisiae yeast cells, and/or wherein the yeast single cell protein product does not comprise Saccharomyces cerevisiae yeast cells.

4. The animal feed of claim 1, wherein said single cell protein product comprises ethanol fed Saccharomycesales yeast cells.

5. The animal feed of claim 1, wherein the Saccharomycesales yeast cells are yeast cells from the genus Cyberlindnera, Kluyveromyces, Wickerhamomyces, Pichia or Yarrowia, preferably from the genus Cyberlindnera or Kluyveromyces or Wickerhamomyces,
preferably wherein the Saccharomycesales yeast cells are from Cyberlindnera jadinii, Kluyveromyces lactis, Wickerhamomyces anomalus, Pichia anomala or Yarrowia lipolytica, preferably from Cyberlindnera jadinii, Kluyveromyces lactis, Wickerhamomyces anomalus,
preferably wherein the Saccharomycesales yeast cells are or are derived from Cyberlindnera jadinii ATCC 26387, Cyberlindnera jadinii FERM-BP1656, Wickerhamomyces anomalus IFO 569, Wickerhamomyces anomalus CBS 1980, Cyberlindnera jadinii ATCC 9950, Kluyveromyces lactis CBS 2896, Wickerhamomyces anomalus CBS 2576 or Yarrowia lipolytica CBS 7504, preferably from Cyberlindnera jadinii ATCC 26387, Cyberlindnera jadinii FERM-BP1656, Wickerhamomyces anomalus IFO 569, Wickerhamomyces anomalusCBS 1980, Cyberlindnera jadinii ATCC 9950 or Kluyveromyces lactis CBS 2896.

6. The animal feed of claim 1, wherein said Saccharomyces yeast cells are capable of producing with ethanol as carbon source 34% (w/w) or more protein per gram dry weight of said Saccharomyces yeast cells, preferably 41% (w/w) or more protein per gram dry weight of said Saccharomyces yeast cells, more preferably 42.5% (w/w) or more protein per gram dry weight of said Saccharomyces yeast cells.

7. The animal feed of claim 1, wherein said yeast single cell protein product comprises all essential amino acids.

8. The animal feed of claim 1, wherein said Saccharomyces yeast cells are not genetically engineered.

9. The animal feed of claim 1, wherein said yeast single cell protein product comprises 34% (w/w) or more protein per gram dry weight of Saccharomyces yeast cells, preferably 41% (w/w) or more protein per gram dry weight of Saccharomycesles yeast cells, more preferably 42.5% (w/w) or more protein per gram dry weight of Saccharomyces yeast cells.

10. The animal feed of claim 1, wherein said yeast single cell protein product comprises dried Saccharomycesales yeast cells., preferably wherein said dried Saccharomyces yeast cells are intact or disrupted or a mixture of intact and disrupted cells.

11. The animal feed of claim 1, wherein i) said animal feed further comprises less than 25% (w/w) plant-based protein products, and/or wherein ii) said animal feed further comprises 5% (w/w) or less fish meal, and/or wherein iii) said animal feed comprises no fish meal, and/or wherein iv) said animal feed comprises from 1 to 25% (w/w) oil.

12. The animal feed of claim 1, wherein said animal feed is a feed for pigs, cows, or sheep or wherein said animal feed is a feed for aquatic species, wherein said aquatic species are selected from crustaceans or fish, preferably wherein said crustaceans are shrimps and/or preferably wherein said fish are warm water fish or cold water fish, preferably wherein said warm water fish are selected from tilapia, seabream, seabass, or carp and/or preferably wherein said cold water fish are selected from salmon or rainbow trout.

13. Use of the animal feed of claim 1 for feeding an animal.

14. Use of the animal feed of claim 1 for increasing body weight of an animal.

15. The use of claim 13, wherein said animal is a pig, cow, or sheep or wherein said animal are aquatic species, preferably wherein said aquatic species are selected from crustaceans or fish, preferably wherein said crustaceans are shrimps and/or preferably wherein said fish are warm water fish or cold water fish, preferably wherein said warm water fish are selected from tilapia, seabream, seabass, or carp and/or preferably wherein said cold water fish are selected from salmon or rainbow trout.
